(19) United States
(12) Patent Application Publication
(10) Pub. No.: US 2026/0249208 A1
(43) Pub. Date: Aug. 27, 2026

(54) METHODS FOR OPTIMIZATION OF DISTILLATION COLUMN FOR REDUCTION OF ENERGY CONSUMPTION

(71) Applicant: SABIC Global Technologies B.V., Bergen OP Zoom (NL)

(72) Inventor: Mahesh Kumar Srinivas, Jubail (SA)

(21) Appl. No.: 18/714,227

(22) PCT Filed: Nov. 30, 2022
(86) PCT No.: PCT/IB2022/061612

§ 371 Date: May 29, 2024

Related U.S. Application Data
(60) Provisional application No. 63/264,675, filed on Nov. 30, 2021.

(51) Int. Cl. B01D 3/42; B01D 3/00; B01D 3/14; B01D 3/32; B01D 5/00; C07C 7/04

(52) U.S. Cl. CPC B01D 3/4216; B01D 3/007; B01D 3/14; B01D 3/32; B01D 5/006; B01D 5/009; C07C 7/04

(57) ABSTRACT

A system and methods for distillation and recovery of ethylene from a cracked hydrocarbon feed material, including a fractional distillation column having a series of trays and multiple distillation outlets, each located at a different tray location or level along the fractional distillation column. The fractional distillation column is controlled to optimize the output of ethylene, including altering operation of the fractional distillation column to enable extraction of streams of ethylene of different selected purity levels and in different phases from the fractional distillation column.

### CROSS-REFERENCE TO RELATED APPLICATIONS

This application claims the benefit of and priority to U.S. Provisional Application No. 63/264,675, filed on Nov. 30, 2021, which is incorporated herein by reference in its entirety.

### TECHNICAL FIELD

The present disclosure generally relates to separation and recovery of high purity ethylene, and in particular to systems and methods of optimizing operation of a fractional distillation column for processing a cracked hydrocarbon feed to recover ethylene of selected high ethylene contents or purities, while achieving a reduction in energy for operation of the fractional distillation column.

### BACKGROUND

As a part of the process for fractionation and production of high purity ethylene, light paraffin compounds such as ethane, propane, butanes, pentanes, naphtha, and or other hydrocarbon effluent or feed are cracked at high temperatures in a furnace, and a hydrocarbon feed or effluent material including ethane and ethylene is supplied to a fractional distillation column for extraction and recovery of high purity ethylene. In the fractional distillation column, the incoming hydrocarbon feed is subjected to a separation process in which the hydrocarbon feed is heated to temperatures sufficient to cause a separation of ethylene from ethane and other constituent materials of the hydrocarbon feed. During the recovery process, ethylene having a purity of at least 99.95 wt. % ethylene is discharged from the fractional distillation column. The discharged high purity ethylene typically is sent to an ethylene product grid or storage tank for sale to consumers for use in production of polypropylene (PP), polyethylene materials (PE), ethylene glycol (EG), vinyl chloride monomers (VCM), and other uses. Due to the close boiling points of ethane and ethylene, the energy required for the separation of ethylene from ethane is one of the major energy consuming steps in the overall production process requirements, and can constitute up to approximately 10% of the total energy requirements in the process to obtain high ethylene purity levels of at least 99.95 wt. %. Moreover, most ethylene recovery systems and processes generally are operated to produce the highest purity level ethylene that is required by most consumers, though such highest purity levels may not be required by some consumers.

Accordingly, it can be seen that a need exists for a system and method for optimizing operations of a fractional distillation column for processing of a cracked hydrocarbon feed to recover ethylene of selected high ethylene content levels or purities that addresses the foregoing and other related and unrelated problems in the art.

### SUMMARY

Briefly described, the present disclosure relates to systems and methods for distillation and recovery of ethylene from cracked hydrocarbon feed materials using a fractional distillation column. The fractional distillation column and the operation thereof are modified and/or controlled to optimize the output of ethylene, including altering operation of the fractional distillation column to enable extraction and output of streams of ethylene of different selected purity levels and in different phases from the fractional distillation column, while further enabling a reduction in energy consumption of the fractional distillation column.

In one aspect, a fractional distillation column is provided, including a plurality of trays stacked within a chamber of a fractional distillation column housing. The fractional distillation column includes an inlet through which a cracked hydrocarbon feed or effluent is received, and will include multiple distillation outlets through which outlet draws or streams of ethylene, including streams of different high purities or high ethylene contents are extracted from the fractional distillation column. The fractional distillation column is connected to a reboiler that supplies heat to the fractional distillation column for the process of separation of ethylene from ethane and other constituent components of the hydrocarbon feed; and further is connected with a condenser that receives a vent stream from the fractional distillation column, the vent stream consisting of vapor containing a lower content or purity of ethylene and additional by-products or constituent materials such as methane and hydrogen. The condenser will condense at least a portion of the vent stream and feed a recycled liquid containing ethylene and other materials such as methane and hydrogen condensed from the vent stream back to the fractional distillation column for further processing and recovery of ethylene therefrom.

In embodiments, the distillation outlets of the fractional distillation column include at least a first outlet and a second outlet each located at a selected tray location or level along the fractional distillation column, and at which first and second ethylene fractions or streams of ethylene, each having a selected ethylene content or purity, are extracted. A third outlet through which the vent stream is vented to the condenser is provided along the fractional distillation housing, generally positioned at an elevation above the first and second outlets. A fourth outlet along the fractional distillation column feeds ethane and other by-product or constituent materials such as methane and/or hydrogen remaining after extraction of the ethylene, to the reboiler, a portion of which is to generate heat while the rest will be recycled back to the furnace.

In some aspects, operation of the fractional distillation column is controlled and altered according to embodiments of the methods disclosed herein. A cracked hydrocarbon feed is supplied from the downstream furnace into the chamber of the fractional distillation column, and is heated to temperatures sufficient to separate ethylene from ethane and other constituent materials in the hydrocarbon feed. The first outlet generally is located along the fractional distillation at a first tray location or level selected for extraction or draw off of a first ethylene fraction or stream, and the second outlet is positioned at a second tray location or level selected for extraction or draw off of a second ethylene fraction or stream. In embodiments, the first ethylene fraction will comprise a liquid phase of ethylene having a first ethylene content or purity of at least approximately 99.95 wt. % ethylene. The second ethylene fraction will be extracted as a vapor or in a vapor phase, and will have a second ethylene content that is different from the first ethylene content, and which is at least approximately 99.90 wt. % ethylene. The third outlet is located at the top of the fractional distillation column and is configured to discharge or vent additional vapor containing lower purity ethylene and by-products or constituent components of the hydrocarbon feed, such as hydrogen, methane and ethane, from the fractional distillation column to the condenser for recycling of at least a portion of the vent stream as a recycled liquid that is fed back to the fractional distillation column. In embodiments, the second outlet can be located adjacent the third outlet through which the vent stream is discharged from the fractional distillation column and fed to the condenser; and in some embodiments, the second outlet can be located along a conduit between the condenser and the fractional distillation column.

In embodiments, the selection of the second tray location for the draw off or extraction of the second ethylene fraction from the fractional distillation column will be determined in conjunction with the selection of the first tray location in order to enable consistent draw off or extraction of the different streams of ethylene having different desired ethylene contents or purities for both the first ethylene fraction, which is extracted or drawn off in a liquid phase, and the second ethylene fraction, which is drawn off or extracted in a vapor phase. Operation of the fractional distillation column will be controlled based on a composition of the incoming hydrocarbon feed, e.g. by controlling the reboiler to control heating of the hydrocarbon feed within the fractional distillation column, so that the desired ethylene contents or purities for the first and second ethylene fractions are achieved and will be available for draw off at the selected first and second tray locations. As a result, the multiple streams of ethylene of different purities can be extracted from the fractional distillation column in different phases (e.g. as liquid and as a vapor) that can be used by different processes and consumers, including processes and consumers that don't necessarily require the highest purity ethylene content. For example, the second ethylene fraction having an ethylene content of 99.90 wt. % ethylene or greater can be provided as a vapor directly to a VCM process, while the first ethylene fraction having an ethylene content at 99.95% ethylene or greater can be sent as a liquid to an ethylene grid or storage for use by PP, PE, EG or other processes.

As a further result of the extraction of the second ethylene fraction in a vapor phase, an increased volume or amount of vapor can be removed from the fractional distillation column, resulting a reduced volume of vented vapor being sent to the condenser. This reduction in volume of the vented vapor can reduce the amount of vapor discharged to the condenser to be recycled, thus enabling a reduction of the heat duty on the condenser, and can further reduce an amount of recycled liquid returned to the fractional distillation column. As a result, power consumption by the condenser and the reboiler can be reduced in a range of approximately 2%-5% up to approximately 10% while a capacity and/or through-put volume of the hydrocarbon feed that can be processed through the fractional distillation column can be increased.

According to additional aspects, a method for separating ethylene from a cracked hydrocarbon feed is provided, the method comprising supplying a cracked hydrocarbon feed to a fractional distillation column having a series of stacked trays; separating ethylene from the hydrocarbon feed; extracting a first ethylene fraction from the hydrocarbon feed at a first tray location or level along the fractional distillation column, the first ethylene fraction being extracted in a liquid phase and having a first ethylene content; extracting a second ethylene fraction from the hydrocarbon feed at a second tray level or location along fractional distillation column, the second ethylene fraction being extracted in a vapor phase and having a second ethylene content, wherein the second ethylene content is different from the first ethylene content; and wherein the first and second tray levels or locations for extracting the first and second ethylene fractions are selected to optimize extraction of the first ethylene fraction in a liquid phase having the first ethylene content or purity and extraction of second ethylene fraction in a vapor phase having the second ethylene content or purity; and altering operation of the fractional distillation column based upon an ethylene percentage by mass of the hydrocarbon feed supplied to the fractional distillation column, to adjust the separation of ethylene of different ethylene contents from the hydrocarbon feed within the fractional distillation column such that the first ethylene content of the first ethylene fraction extracted in liquid phase at the first tray level or location comprises at least approximately 99.95 wt. % ethylene, and the second ethylene content of the second ethylene fraction extracted in vapor phase at the second tray level or location is at least approximately 99.90 wt. % ethylene.

In embodiments, the method further comprises discharging a vent stream in a vapor phase to a condenser and a waste stream in a liquid phase to a reboiler.

In embodiments, the second ethylene fraction is extracted from the fractional distillation column at a location adjacent an outlet through which the vent stream is discharged to the condenser. In some embodiments, a volume of vapor within the fractional distillation column to be discharged as the vent stream discharged to the condenser is at least partially reduced by the extraction of the second ethylene fraction from the fractional distillation column.

In embodiments, of the method, a heat duty of the condenser is reduced by approximately 1.0 MW to approximately 4.0 MW, and a heat duty of the reboiler is reduced by approximately 0.1 MW to approximately 0.5 MW. In some embodiments, an overall heat duty used by the fractional distillation column is reduced by approximately 0.5 MW to approximately 2.0 MW.

In embodiments, the second tray location or level is at a higher elevation along the fractional distillation column than the first tray level.

In embodiments, the method further comprises supplying the second ethylene fraction in its vapor phase to a vinyl chloride monomer (VCM) process or similar processes capable of utilizing an ethylene content about of 99.90 wt. %.

In embodiments, the method further comprises supplying the first ethylene fraction to one or more of a storage tank or an ethylene grid to supply to one or more ethylene consumers.

In embodiments, separating the first ethylene fraction and the second ethylene fraction from the hydrocarbon feed reduces energy consumption by at least about 5% in embodiments, energy consumption resulting from operation of one or more of a condenser to which the vent stream is supplied or a reboiler associated with the fractional distillation column is reduced by at least approximately 5%.

In embodiments, the hydrocarbon feed comprises ethane, ethylene and one or more of naphtha, methane, propane, propylene, butanes, pentanes, and hydrogen.

According to other aspects of the present disclosure, a fractional distillation column is provided for separation of ethylene from a cracked hydrocarbon feed, the fractional distillation column comprising a chamber, a plurality of trays stacked at varying elevations with the chamber; at least one inlet configured to receive the hydrocarbon feed; and a plurality of outlets, including at least a first distillation outlet and a second distillation outlet each positioned along the fractional distillation column at a selected one of a plurality of trays within the fractional distillation column, wherein the first distillation outlet is positioned at a first tray location or level selected to extract a first ethylene fraction in a liquid phase separated from the distillation hydrocarbon feed, the first ethylene fraction having a first ethylene content of at least 99.95 wt. % ethylene, and the second outlet is positioned at a second tray location or level at a higher elevation or position along the fractional distillation column than the first tray location or level and selected to extract a second ethylene fraction in a vapor phase separated from the hydrocarbon feed, the second ethylene fraction having a second ethylene content of at least 99.90 wt. % ethylene. A third outlet is positioned at a third location or elevation along the fractional distillation column, generally an upper end thereof; the third outlet configured to discharge a vent stream of vapor from a volume of vapor generated within the fractional distillation column to a condenser; wherein the condenser is configured to condense at least a portion of the vent stream and return a recycled liquid stream containing ethylene and one or more constituent materials including hydrogen and/or methane to the fractional distillation column; wherein the volume of vapor available for discharge to the condenser as the vent stream is at least partially reduced by the extraction of the second ethylene fraction such that a heat load on at least one of the condenser or a reboiler connected to the fractional distillation column is reduced sufficient to reduce a total energy consumption of the fraction of distillation column by approximately 0.5 MW to approximately 2.0 MW.

In embodiments of the fractional distillation column has approximately 100-150 trays at successively higher elevations within the chamber.

In embodiments, reduces energy consumption resulting from operation of one or more of the condenser to which the first ethylene fraction is supplied or (2) a reboiler associated with the fractional distillation column is reduced energy consumption by at least approximately 5% or greater.

In embodiments of the fractional distillation column, the first ethylene fraction is supplied to one or more of a storage tank or an ethylene grid to supply to one or more ethylene consumers, and the second ethylene fraction can be supplied in its vapor phase directly to a vinyl chloride monomer (VCM) process.

In embodiments, the fractional distillation column, further comprises a fourth outlet configured to feed a waste stream including methane and hydrogen to a reboiler associated for generation of heat within the fractional distillation column.

In embodiments, the cracked hydrocarbon feed comprises ethane, ethylene and one or more of naphtha, methane, propane, propylene, butanes, pentanes, and hydrogen; and wherein the heat supplied to the fractional distillation column is adjusted based upon an ethylene content of the incoming hydrocarbon feed to control separation of the ethylene therefrom such that the ethylene separated from hydrocarbon feed at the first tray location comprises ethylene in its liquid phase having the first ethylene content, and the ethylene separated from the hydrocarbon feed at the second tray location comprises ethylene in its vapor phase having the second ethylene content.

Accordingly, embodiments of systems and methods for distillation and recovery of ethylene from hydrocarbon feed materials using a fractional distillation column, wherein the fractional distillation column and the operation thereof is modified and/or controlled to optimize the output of ethylene, that are directed to the above discussed and other aspects are disclosed. The foregoing and other aspects of the present disclosure will become apparent and more readily appreciated from the following detailed description, taken in conjunction with the accompanying drawings. Moreover, it is to be understood that both the foregoing summary of the disclosure and the following detailed description are exemplary and intended to provide further explanation without limiting the scope of the present disclosure.

### BRIEF DESCRIPTION OF THE DRAWINGS

The accompanying drawings, which are included to provide a further understanding of the embodiments of the present disclosure, are incorporated in and constitute a part of this specification, illustrate embodiments of this disclosure, and together with the detailed description, serve to explain the principles of the embodiments discussed herein. No attempt is made to show structural details of this disclosure in more detail than may be necessary for a fundamental understanding of the exemplary embodiments discussed herein and the various ways in which they may be practiced.

FIG. 1 is a schematic illustration of an example of a process for production of ethylene.

FIG. 2 shows a fractional distillation column, schematically illustrating a method of operation thereof, according to embodiments of the present disclosure.

FIG. 3 is a graphical illustration showing heat duty of a condenser and a reboiler in relation to ethylene content and a selection of tray locations for extraction of ethylene fractions of varying ethylene content.

### DETAILED DESCRIPTION

The foregoing description generally illustrates and describes various embodiments of the present invention. It will, however, be understood by those skilled in the art that various changes and modifications can be made to the above-discussed construction of the present invention without departing from the spirit and scope of the invention as disclosed herein, and that it is intended that all matter contained in the above description or shown in the accompanying drawings shall be interpreted as being illustrative, and not to be taken in a limiting sense. Furthermore, the scope of the present disclosure shall be construed to cover various modifications, combinations, additions, alterations, etc., above and to the above-described embodiments, which shall be considered to be within the scope of the present invention. Accordingly, various features and characteristics of the present invention as discussed herein may be selectively interchanged and applied to other illustrated and non-illustrated embodiments of the invention, and numerous variations, modifications, and additions further can be made thereto without departing from the spirit and scope of the present invention as set forth in the appended claims.

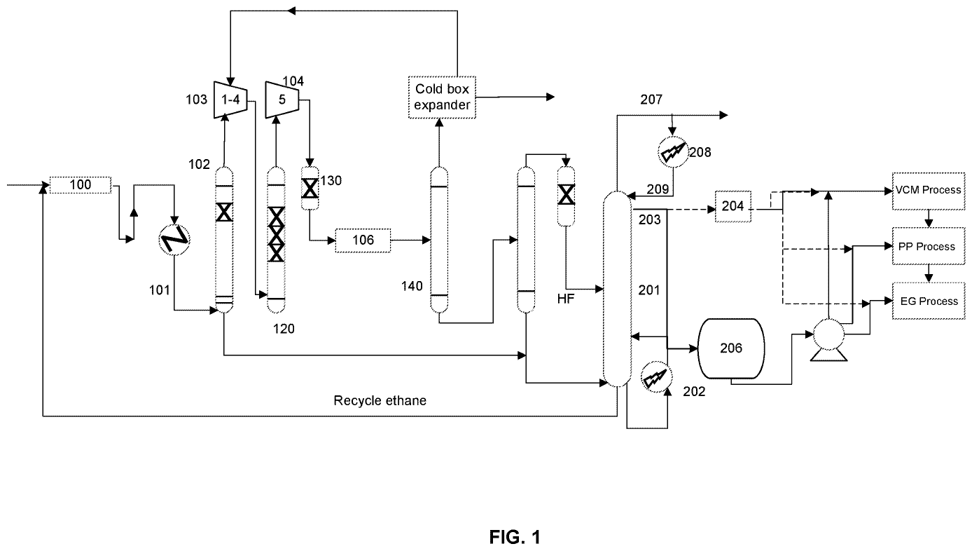

FIG. 1. schematically illustrates a prior art process and system for production of high purity ethylene having an ethylene content of at least 99.95 wt. % ethylene. As shown in FIG. 1, hydrocarbon materials are fed into a furnace 100 that produces an output 101 hydrocarbon material that is fed into a quench tower 102 that cools the incoming heated hydrocarbon material and produces an output gas 103 that is fed to a compressor 104 that compresses the hydrocarbon gas. The condensed hydrocarbon material is then passed through one or more chillers 106 that chill the hydrocarbon gas to form a cracked hydrocarbon feed or efficient material (HF) vapor fed to a downstream fractional distillation column 201. As indicated in FIG. 1, the hydrocarbon feed material output from the compressor also can be passed through other processing stations such as a caustic tower 120, dryer 130, a demethanizer 140, and other processes to refine the cracked hydrocarbon feed material or effluent prior to its introduction into the separating unit 200.

As further indicated in FIG. 1, the cracked hydrocarbon feed HF is input into the separating unit, which generally comprises a fractional distillation column or C2 splitter 201, coupled to a reboiler 202. The cracked hydrocarbon feed is subjected to heating to cause separation of high purity ethylene from additional constituent components or byproducts of the hydrocarbon feed, such as ethane, methane, hydrogen and other constituent components. As indicated at 203, high purity ethylene having an ethylene content of at least 99.95 wt. % ethylene is drawn off from the fractional distillation column and can be provided to an ethylene grid 204 through which the high purity ethylene is supplied to downstream processes such as a vinyl chloride monomer (VCM) process or other processes such as a (PP) process polypropylene, polyethylene (PE) process, or ethylene glycol (EG) process. Alternatively, as further indicated in FIG. 1, the high purity ethylene can be sent to an ethylene product storage tank 206 and for storage and later supply to the VCM, PP, PP, and/or EG processes. Vapor is vented, as indicated at 207 to a condenser 208 that condenses the vapor and returns a recycled liquid stream 209 to the fractional distillation column.

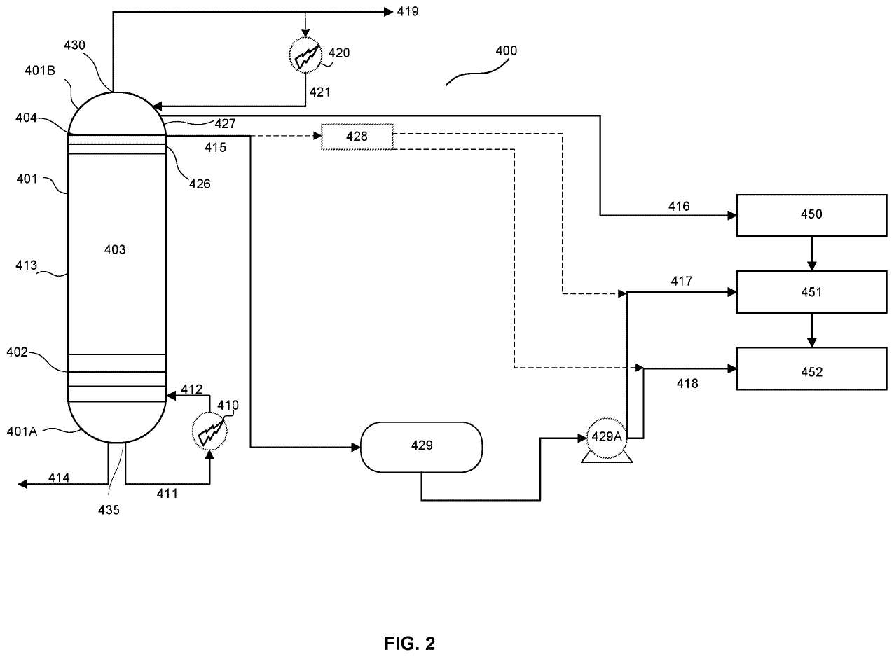

FIG. 2 illustrates an embodiment of a system 400 for separation and recovery of ethylene of different high purities or content. The system includes a fractional distillation column 401 configured and operated according to embodiments of the present disclosure. As indicated in FIG. 2, the fractional distillation column or C2 splitter 401 generally will include a housing 402 defining a distillation chamber 403 housing a series of trays 404 stacked vertically therein. In embodiments, the fractional distillation column can have between approximately 100 to 150 trays. However, in other embodiments, greater or lesser numbers of trays also can be used. An inlet will be defined along the fractional distillation column 401, for example, as indicated at 413, and will supply a cracked hydrogen feed or effluent material HF into the chamber 403 of the fractional distillation column. The hydrogen feed introduced into the fractional distillation column generally will be introduced in a vapor phase, e.g., at temperatures below approximately −10° C. to −12° C., and is subjected to heating sufficient to cause a separation of ethylene from ethane and other constituent components of the hydrogen feed within the fractional distillation column.

As further illustrated in FIG. 2, the fractional distillation column 401 will be coupled to and in communication with a reboiler 410, generally located adjacent the bottom or lower end 401A of the fractional distillation column 401. The reboiler will receive a liquid waste stream 411, for example comprising ethane, hydrogen, methane, and other constituent components of the hydrocarbon feed separated from the ethylene. A portion of the waste stream can be used by the reboiler to generate heat supplied to the chamber of the fractional distillation column indicated by return 412. Other materials, such as hydrogen and ethane, that have been removed from the hydrocarbon feed and separated from the ethylene can be removed from fractional distillation column as indicated at 414 and routed back to the furnace (FIG. 1) for continued processing.

As further illustrated in FIG. 2, a condenser 420 is in communication with the fractional distillation column 401 and receives a vent stream indicated at 419 (e.g. via conduit) comprising a vapor including ethylene, hydrogen and methane separated from the hydrocarbon feed. The ethylene content of the vent stream 419 typically is below a purity level that is required by downstream consumers, e.g. PP, EG, PE and/or VCM processors. The condenser 420 condenses at least a portion of the vent stream and returns a recycled liquid captured from the condensing of the vent stream back into the chamber of the fractional distillation column for continued processing and recovery of ethylene therefrom as indicated at 421. The fractional distillation column further includes a series of distillation outlets 425 located at varying tray locations or elevations along the fractional distillation column as indicated in FIG. 2.

In embodiments, the distillation outlets of the fractional distillation column 401 generally will include at least a first outlet 426 configured for extraction or draw off of and delivery of a first ethylene fraction or stream 415 of high purity ethylene in a liquid phase (e.g. the DPRODUCT of Table 2 below); and a second outlet 427 configured to extract or draw off of a second ethylene fraction 416 of a different selected purity or ethylene content and which is extracted in a vapor phase from the fractional distillation column. As further shown in FIG. 2, the second ethylene fraction or stream 416 can be supplied in its vapor phase directly from the fractional distillation column through the second outlet 427 to a downstream VCM process 450 or other similar process that typically does not require the highest purity or content ethylene (e.g. having an ethylene content of at least 99.95 wt. % ethylene); while the first ethylene fraction can be fed to an ethylene grid 428 or to a storage tank 429 for supply to a PP/PE process 451 or similar process or consumer as shown at 417, or from the storage tank by a pump 429A to an EG process 452 or similar process or consumer as indicated at 418.

In addition, a third outlet 430 generally will be provided at the upper end or top 401B of the fractional distillation column, the third outlet being configured to discharge the vent stream 419 to the condenser 420, which condenses a portion of the vent stream and sends recycled liquid captured therefrom back to the fractional distillation column, as indicated at 421, while a portion of the vent stream can be vented or sent to further, downstream processing. A fourth outlet 435 generally is provided at the lower end of the fractional distillation column for discharging a waste stream to the reboiler 410.

The hydrocarbon feed or effluent HF supplied to the fractional distillation column 401 generally will be comprised of ethylene and ethane as well as additional components or byproducts such as hydrogen, methane, propylene and propane in varying weight percentages. The composition of the incoming hydrocarbon feed generally will be monitored or determined, and a range of ethylene percentages of the incoming hydrocarbon feed, as well as historical measurements of incoming hydrocarbon feeds supplied to the fractional distillation column, can be used to generate a range or estimated average amount of an ethylene percentage for incoming hydrocarbon feeds.

The incoming hydrocarbon feed input into the chamber of the fractional distillation column generally will be in a vapor phase and at a temperature of approximately −10° C. to −12° C. or lower. The reboiler is controlled to supply heat to the chamber of the fractional distillation column sufficient to cause the ethylene present within the hydrocarbon feed to approach its boiling point and separate from the ethane and other constituent components of the hydrocarbon feed such as methane and hydrogen. As the ethylene is heated to its boiling point, at least a portion of the ethylene is transformed into a liquid phase and is separated from the ethane present within the hydrocarbon feed, and generally will include high purity ethylene having an ethylene content of at least 99.95 wt. %. As the liquid ethylene is separated from the ethane of the hydrocarbon feed remaining vapor of the hydrocarbon feed continues to rise along the fractional distillation column, which vapor includes ethylene, ethane and constituent components elements such as hydrogen and methane and has a lower purity or ethylene content e.g., an ethylene content of lower than 99.95 wt. % ethylene.

As further indicated in FIG. 2, the distillation outlets 425 of the fractional distillation column of the present disclosure are configured to enable draw off or extraction of ethylene streams having different purity levels or ethylene content, and extract such ethylene streams in different phases, e.g. in a liquid phase and in a vapor phase. The first outlet generally will include a fluid control valve configured to extract or draw off and/or discharge a liquid ethylene stream. The first outlet further will be located along the fractional distillation column first at a tray location or level selected as a tray location or level at which the draw off or extraction of a first ethylene content of at least 99.95 wt. % ethylene is optimized for range of ethylene percentages of the incoming hydrocarbon feed.

The second outlet will be located at a second tray location or level that is at an elevation above or higher along the fractional distillation column than the first outlet. The second outlet generally will be configured for extraction or draw off of the second ethylene fraction having a second ethylene content that is less than the first ethylene content of 99.95 wt. % ethylene, but is at least 99.90 wt. % ethylene and is in a vapor phase. The location of the second outlet at a second tray location or level will be determined similarly to the location of the first outlet at the first tray location or level based upon a determination of an optimum tray level or location within a range of tray levels or locations at which ethylene is vapor phase having an ethylene content that meets or exceeds the second ethylene content of at least 99.90 wt. % ethylene.

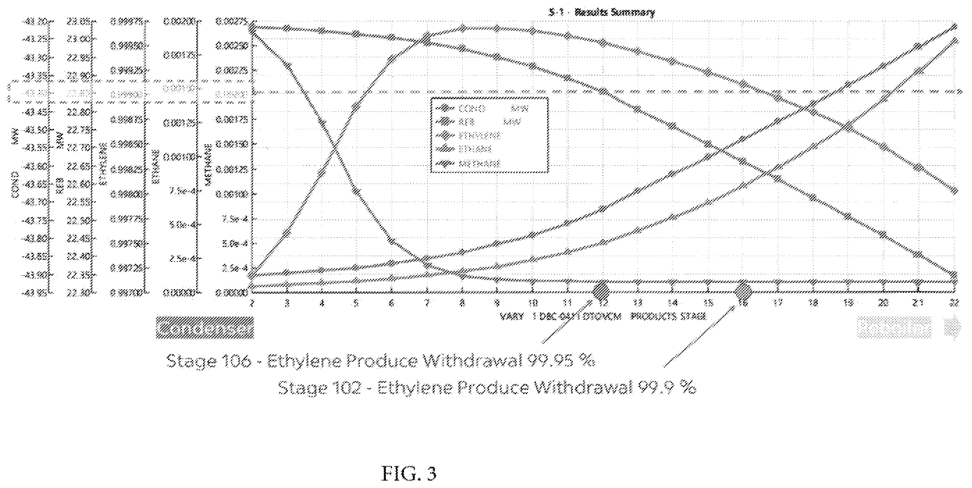

FIG. 3 graphically illustrates a selection of the first and second tray locations or level at which extraction or recovery of the first and second ethylene fractions or streams in their liquid and vapor phases will be recovered from an incoming hydrocarbon feed with a known ethylene percentage. By way of example, if an average or range of ethylene percentages of incoming hydrogen feeds supplied to the fractional distillation column falls within a range of 60%-80%, the first and second tray locations for extraction of the desired high purity ethylene of at least 99.95 wt. % in a liquid phase ethylene and lower purity ethylene of at least 99.90 wt. % ethylene, can be determined as optimal tray locations or levels within a projected range of tray locations or levels for draw off or extraction of the selected ethylene purities or content.

With the optimum first and second tray locations or levels determined for location of the first and second outlets, the operation of the fractional distillation column will be controlled or altered, e.g. by manipulating heat input into the chamber thereof, in order to adjust the ethylene content or purity levels of the liquid and vapor phases of ethylene separated from the hydrocarbon feed within the fractional distillation column so that the first ethylene content of the first ethylene fraction extracted from the fractional distillation column in liquid form through the first outlet at the first tray location or level will have a purity or ethylene content of at least 99.95 wt. % ethylene, and additionally that the second ethylene content of the second ethylene fraction extracted or drawn off from the fractional distillation column through the second outlet at the second tray location or level as a vapor will be at least 99.90 wt. % ethylene.

By adjusting the operation of the fractional distillation column based upon the ethylene composition of the incoming hydrogen feed, multiple output streams of ethylene, each having a different purity, including at least one high purity ethylene stream with an ethylene content or purity of at least 99.95 wt. % ethylene, and at least one ethylene stream having an ethylene content of at least 99.90 wt. % ethylene, can be extracted. The first ethylene fraction 415 extracted liquid phase has the highest ethylene purity and can be supplied to the ethylene grid 428 or to a product storage tank 429 for supply to processes or consumers that acquire the highest purity ethylene, such as for PP, PE, EG processes for production of polypropylene, polyethylene, ethylene glycol, or other products. The second ethylene fraction 416 that is drawn off as a vapor from the fractional distillation column, and which has a lower ethylene content and the first ethylene fraction, but which is still at least 99.90 wt. % ethylene, can be supplied as a vapor directly to consumers or processes such as a VCM process or other consumer or process that does not require the highest purity ethylene.

The second outlet generally is located adjacent towards the upper or top end of the fractional distillation column, typically being near or adjacent the third outlet through which the vent stream is exhausted or discharged from the fractional distillation column and fed to the condenser. In some embodiments, the second outlet can be located adjacent the third outlet or along a conduit or flow path between the fractional distillation column and the condenser. The vent stream discharged from the third outlet generally is discharged as a vapor having a lower content or purity level of ethylene, e.g. below 99.90 wt. % ethylene, with higher levels of additional components or constituents, such as hydrogen and methane which generally are returned to the fractional distillation column as part of the recycled liquid from the condenser. By removing the second ethylene fraction or stream with an ethylene content of at least 99.90 wt. % ethylene, a volume of vapor with an ethylene content of below 99.90% wt. % ethylene remaining within the chamber of the fractional distillation column and that needs to be vented to the condenser can be reduced. By lowering the volume of the vent stream that is fed to and that needs to be condensed or processed by the condenser, the heat load or duty on the condenser can be reduced, sufficient to help provide a reduction in overall energy consumption of the fractional distillation column. In addition, the reduction in the volume of the vent stream processed by the condenser further can lead to a reduction of the amount or volume of recycled liquid fed back into the fractional distillation column, which can help reduce the heat duty on the reboiler, and further can enable the fractional distillation column to increase its capacity for the incoming hydrocarbon feed so as to increase throughput processing thereof. In embodiments, capacity of the fractional distillation column can be increased by 1% to 10% and potentially higher.

Table 1 set forth below illustrates an example comparing operating conditions of a conventional distillation arrangement against operating conditions of the optimized system and methods for distillation of ethylene to enable extraction of multiple ethylene streams of different ethylene content or purity in accordance with the present disclosure. The comparative studies were generated using a process simulation tool, an example of which is ASPEN Plus® from Aspen Technologies, Inc. The feed rates, temperature and the pressures for the incoming hydrocarbon feeds for each case study were kept constant for the both case studies, while the ethylene stream draw off trays for the ethylene streams extracted/generated in each case were varied.

TABLE 1
Comparison of Condenser and Reboiler Performance:
CURRENT CASE

| Condenser Duty | | | Reboiler Duty | | |
|---|---|---|---|---|---|
| Name | Value | Units | Name | Value | Units |
| Temperature | −32.42 | C. | Temperature | −4.42 | C. |
| Heat duty | −45.60 | MW | Heat duty | 23.50 | MW |
| Distillate rate | 0.67 | tonne/hr | Bottoms rate | 28.91 | tonne/hr |
| Reflux rate | 496.78 | tonne/hr | Boilup rate | 266.57 | tonne/hr |
| Reflux ratio | 741.46 | | Boilup ratio | 9.22 | |
| | | | Bottoms to | | |
| | | | feed ratio | | |

FUTURE CASE

| Condenser Duty | | | Reboiler Duty | | |
|---|---|---|---|---|---|
| Name | Value | Units | Name | Value | Units |
| Temperature | −29.34 | C. | Temperature | −4.53 | C. |
| Heat duty | −43.89 | MW | Heat duty | 23.02 | MW |
| Distillate rate | 0.67 | tonne/hr | Bottoms rate | 29.04 | tonne/hr |
| Reflux rate | 488.21 | tonne/hr | Boilup rate | 261.04 | tonne/hr |
| Reflux ratio | 728.68 | | Boilup ratio | 8.99 | |

As indicated in Table 1, the heat duty on the condenser using the system and method of the current disclosure can be reduced by approximately 1.0-4.0 megawatts (MW), and, in the example shown above, showed a reduction of approximately 2.07 MW (e.g. from −45.60 MW to −43.89 MW). Similarly, the reboiler duty can be reduced by approximately 0.1 MW-1.5 MW, and in the example shown above, was shown to provide a reduction in the heat duty of the reboiler of approximately 0.95 MW (e.g. from 34.50 MW to 23.02 MW). As a result of the reductions in the heat duty consumed by the reboiler and/or the condenser, power consumption by the fractional distillation column can be reduced by approximately 1.0-3.0 megawatts, and in embodiments, by approximately 1.5-1.7 MW or in a range of at least about 2%-5% or greater, while enabling recovery of multiple streams of ethylene having different ethylene content or purity levels.

Table 2 below shows a comparison of results of a process for extraction of high purity ethylene using a fractional distillation system and process as shown in FIG. 1 versus a simulated example operation of the methods/processes and system for extraction of different streams of ethylene having different ethylene purities or contents and being in different phases can be supplied to different consumers. As indicated in Table 2, the first ethylene fraction or stream—identified as DPRODUCT—is extracted having an ethylene purity of content of at least 99.95 wt. % ethylene, and is extracted without a substantive reduction in purity as compared to the PRODUCT generated using the process of FIG. 1. As also indicated in Table 2, the additional, second ethylene fraction (DTOVCM) is extracted in its vapor phase using the processes of the present disclosure with an ethylene content of 99.91 wt. % ethylene. This DTOVCM ethylene stream or fraction can be supplied directly to a VCM process in its vapor form, since the VCM process does not require a highest purity ethylene content of at least 99.95 wt. % ethylene and can process the lower purity ethylene in its vapor phase, and can be extracted from the fractional distillation column and processes of the present disclosure without a substantive impact on or difference in the purity and recovery of the high purity ethylene (e.g. an ethylene content of 99.95 wt. % ethylene or greater). The third ethylene fraction, indicated by 419, includes a vent stream vented from the fractional distillation column and, generally, is routed to the condenser. As indicated in Table 2 below, the third ethylene fraction will be in a vapor phase and will have a third ethylene content that generally will be less than the first and second ethylene contents of the first and second ethylene fractions, and further will have higher levels of constituent components such as hydrogen and methane. The condenser will condense the third ethylene fraction and return a recycled liquid stream to the fractional distillation column for further processing to remove the ethylene from the hydrogen and methane, with the methane used as fuel for the reboiler and the hydrogen returned to the upstream furnace. The process results in the extraction of multiple ethylene streams of different purities while further providing a reduction in energy consumption of the fractional distillation column by at least 1%-5% or greater.

TABLE 2
Comparison of Material balance

| | | [symbol] | | | | [symbol] | | | |
|---|---|---|---|---|---|---|---|---|---|
| | Units | [symbol] | [symbol] | [symbol] | PRODUCT | [symbol] | [symbol] | [symbol] PRODUCT | [symbol] |
| Phase | | Vapor | Liquid | Vapor | Liquid | Liquid | Vapor | Liquid | Vapor |
| | | Phase | Phase | Phase | Phase | Phase | Phase | Phase | Phase |
| Temperature | C. | −12.80 | −4.42 | −32.42 | [symbol] 26.4 [symbol] | −4.54 | −32.2 [symbol] | −2 [symbol] . [symbol] | −26.44 |
| Pressure | [symbol] | 21.90 | 21.76 | 2 [symbol] .54 | 21. [symbol] | 21. [symbol] | 2 [symbol] . [symbol] | 21. [symbol] | 2 [symbol] . [symbol] |
| Mass Density | kg/ [symbol] | 38.10 | [symbol] 8 [symbol] .26 | 40.72 | 402. [symbol] | 382.41 | 40.72 | 40 [symbol] . [symbol] | 40.75 |
| Average MW | | 28.48 | 30.13 | 27.24 | 28.0 [symbol] | [symbol] 0.12 | 27.27 | [symbol] .0 [symbol] | 2 [symbol] .0 [symbol] |
| [symbol] | | | | | | | | | |
| HYDROGEN | | 0.0024% | 0.0000% | 0.4401% | 0.0000% | 0.0000% | 0. [symbol] % | 0.0000% | 0.0000% |
| METHANE | | 0.0330% | 0.0000% | [symbol] 02% | 0.0027% | 0.0000% | [symbol] . [symbol] % | 0.0028% | 0.0 [symbol] % |
| ETHANE | | [symbol] . [symbol] % | [symbol] . [symbol] % | 0.004% | 0. [symbol] % | [symbol] . [symbol] 23% | 0.004% | 0. [symbol] % | 0. [symbol] % |
| ETHYLENE | | [symbol] .421% | 1. [symbol] % | [symbol] .77 [symbol] % | [symbol] . [symbol] 2% | 1.881% | [symbol] . [symbol] % | 99. [symbol] % | [symbol] . [symbol] 11% |
| PROPYLEN | | 0.1400% | 0. [symbol] 7 [symbol] 0% | 0.0000% | 0.0000% | 0.6699% | 0.0000% | 0.0000% | 0.0000% |
| PROPANE | | 0.00 [symbol] % | 0.0257% | 0.0000% | 0.0000% | 0.02 [symbol] % | 0.0000% | 0.0000% | 0.0000% |
| [symbol] | tonne/hr | 131.44 | 2 [symbol] .01 | [symbol] | 101.82 | [symbol] .04 | 0. [symbol] 7 | [symbol] 7.70 | 14. [symbol] |
| HYDROGEN | tonne/hr | 0.00 | 0.00 | 0.00 | 0.00 | 0.00 | 0.00 | 0.00 | 0.00 |
| METHANE | tonne/hr | 0.02 | 0.00 | 0.02 | 0.00 | 0.00 | 0.02 | 0.00 | 0.00 |
| ETHANE | tonne/hr | 28. [symbol] 0 | 28.25 | 0.00 | 0.05 | 28.25 | 0.00 | 0.04 | 0.01 |
| ETHYLENE | tonne/hr | 102.80 | 0.38 | 0. [symbol] | 101.77 | 0.5 [symbol] | 0. [symbol] | 87. [symbol] | [symbol] 3.99 |
| PROPYLEN | tonne/hr | 0.27 | 0.27 | 0.00 | 0.00 | 0.27 | 0.00 | 0.00 | 0.00 |
| PROPANE | tonne/hr | 0.01 | 0.01 | 0.00 | 0.00 | 0.01 | 0.00 | 0.00 | 0.00 |

[symbol]

indicates data missing or illegible when filed

The present disclosure has been described herein in terms of examples that illustrate principles and aspects of the present disclosure. The skilled artisan will understand, however, that a wide gamut of additions, deletions, and modifications, both subtle and gross, may be made to the presented examples without departing from the spirit and scope of the present disclosure.

What is claimed is:

1. A method for separation of ethylene from a hydrocarbon feed, comprising:
supplying a hydrocarbon feed to a fractional distillation column;
heating the hydrocarbon feed sufficient to cause separation of ethylene from the hydrocarbon feed;
extracting a first ethylene fraction from the hydrocarbon feed at a first location along the fractional distillation column;
extracting a second ethylene fraction from the hydrocarbon feed at a second location along fractional distillation column; and
adjusting separation of the ethylene from the hydrocarbon feed within the fractional distillation column based upon a percentage of ethylene within the hydrocarbon feed supplied to the fractional distillation column such that the first ethylene fraction is extracted in a liquid phase at the first location and comprises a first ethylene content of at least approximately 99.95 wt. % ethylene, and the second ethylene fraction is extracted in a vapor phase at the second location and comprises a second ethylene content of at least approximately 99.90 wt. % ethylene.

2. The method of claim 1, further comprising discharging a vent stream in a vapor phase to a condenser, the vent stream comprising ethylene and at least one of hydrogen and methane; and discharging a waste stream in a liquid phase to a reboiler.

3. The method of claim 2, wherein the second location is positioned adjacent an outlet through which the vent stream is discharged from the fractional distillation column; wherein, the second ethylene fraction is drawn from a volume of vapor formed within fractional distillation column having an ethylene content less than the first ethylene content such that a volume of the vent stream discharged to the condenser is at least partially reduced by the extraction of the second ethylene fraction; and wherein a heat duty of the condenser is reduced by approximately 1.0 MW to approximately 2.0 MW, and a heat duty of the reboiler by approximately 0.1 MW to approximately 0.5 MW.

4. The method of claim 1, wherein energy consumption of the fractional distillation column is reduced by approximately 0.5 MW to approximately 2.0 MW.

5. The method of claim 1, wherein the second location is at a higher elevation than the first location along the fractional distillation column.

6. The method of claim 1, further comprising supplying the second ethylene fraction in its vapor phase to a vinyl chloride monomer (VCM) process.

7. The method of claim 1, further comprising supplying the first ethylene fraction to one or more of a storage tank or an ethylene grid for supply to one or more ethylene consumers.

8. The method of claim 1, wherein separating the first ethylene fraction and separating the second ethylene fraction from the hydrocarbon feed reduces energy consumption of the fractional distillation column by at least about 5%.

9. The method of claim 1, wherein heating the hydrocarbon feed produces a volume of vapor within the fractional distillation column; wherein a first portion of the volume of vapor is extracted as the second ethylene fraction, and further comprising discharging a second portion of the volume of vapor remaining after extraction of the first portion as a vent stream supplied to a condenser, and wherein energy consumption from operation of one or more of the condenser for condensing the vent stream or a reboiler coupled to the fractional distillation column is reduced by at least approximately 5%.

10. The method of claim 1, wherein the hydrocarbon feed supplied to the fractional distillation column comprises ethane, ethylene and one or more of naphtha, methane, propane, propylene, butanes, pentanes, and hydrogen.

11. A method for separating ethylene from a hydrocarbon feed, the method comprising:
supplying a hydrocarbon feed to a fractional distillation column;
separating ethylene in liquid and vapor phases from the hydrocarbon feed within the fractional distillation column;
extracting a first ethylene fraction from the hydrocarbon feed, the first ethylene fraction extracted in a liquid phase, and having an ethylene content of 99.95 wt. % or greater;
extracting a second ethylene fraction from fractional distillation column, the second ethylene fraction extracted in a vapor phase and having an ethylene content of 99.90 wt. % or greater; and
discharging a vent stream from the fractional distillation column to a condenser, the vent stream being discharged in vapor phase and including an ethylene content less than 99.90 wt. %;
wherein extracting the second ethylene fraction reduces a heat duty on the condenser such that energy consumption of the fractional distillation column due to reduction of the heat duty on the condenser is reduced by at least approximately 5%.

12. The method of claim 11, wherein extracting the first ethylene fraction and the second ethylene fraction, comprises drawing the first ethylene fraction from the fractional distillation column at a first level, and drawing the second ethylene fraction from the fractional distillation column at a second level; wherein the second level is higher than the first level.

13. The method of claim 11, further comprising supplying the second ethylene fraction to a vinyl chloride monomer (VCM) process or similar processes that utilizes an ethylene content of 99.0-99.90 wt. %.

14. The method of claim 11, further comprising; supplying the first ethylene fraction to one or more of a storage tank or an ethylene grid for supply to one or more ethylene consumers.

15. The method of claim 11, wherein the hydrocarbon feed comprises ethane, ethylene and one or more of naphtha, methane, propane, propylene, butanes, pentanes, and hydrogen.

16. A system for separation of ethylene from a cracked hydrocarbon feed, the system comprising:
a fractional distillation column comprising:
an inlet positioned to receive the cracked hydrocarbon feed;
a plurality of outlets, each of the plurality of outlets being positioned along the fractional distillation column at one of a plurality of levels and comprising:
a first outlet positioned at a first level of the plurality of levels, the first outlet configured to extract a first ethylene fraction in a liquid phase from the cracked hydrocarbon feed, wherein the first ethylene fraction has a first ethylene content;
a second outlet positioned at a second level of the plurality of levels, the second distillation outlet configured to extract a second ethylene fraction in a vapor phase from the cracked hydrocarbon feed, wherein the second ethylene fraction has a second ethylene content; and
a third outlet, the third distillation outlet configured to extract a third ethylene fraction in a vapor phase from the cracked hydrocarbon feed, wherein the third ethylene fraction has a third ethylene content, the third ethylene content being of a lower ethylene content than the first ethylene fraction and the second ethylene fraction;
a condenser in communication with the third outlet and configured to condense at least a portion of the third ethylene fraction and return a liquid recycled stream including ethylene to the fractional distillation column; and
a reboiler connected to the fractional distillation column and configured to receive a stream of constituent materials of the hydrocarbon feed remaining after separation of ethylene therefrom;
wherein extracting the second ethylene fraction in the vapor phase from fractional distillation column reduces a volume of the third ethylene fraction discharged to the condenser such that energy consumption resulting from operation of one or more of the condenser to which the third ethylene fraction is supplied or the reboiler is reduced by approximately 0.5 MW to approximately 4.0 MW.

17. The system of claim 16, wherein the first level is higher lower than the third level, and the second level is lower than the third level.

18. The system of claim 16, wherein the first ethylene content is 99.95 wt. % ethylene or greater, and the second ethylene content is greater than 99.90 wt. % ethylene.

19. The system of claim 16, wherein separating the third ethylene fraction from one or more of the first ethylene fraction or the second ethylene fraction reduces energy consumption of the fractional distillation column by 5% or greater.

20. The system of claim 16, wherein the first outlet is configured to supply the first ethylene fraction in liquid phase to one or more of (1) a storage tank or (2) an ethylene grid to supply to one or more ethylene consumers that utilize ethylene contents of 99.95 wt. % ethylene or greater; and the second outlet is configured to supply the second ethylene fraction in the vapor phase to a vinyl chloride monomer (VCM) process.

21. A method for recovery of ethylene, the method comprising:
supplying a cracked hydrocarbon feed to a fractional distillation column;
separating a first ethylene fraction from the cracked hydrocarbon feed, the first ethylene fraction being a liquid phase and having an ethylene content of 99.95 wt. % ethylene or greater;
separating a second ethylene fraction from the cracked hydrocarbon feed, the second ethylene fraction being in a vapor phase and having an ethylene content of 99.90 wt. % ethylene;
separating a third ethylene fraction from the cracked hydrocarbon feed, the third ethylene fraction being in vapor phase and having a third ethylene content that is different from the first and second ethylene contents;
wherein separating the first ethylene fraction in liquid phase and the second ethylene fractions and third ethylene fraction in vapor phases with different ethylene contents from the cracked hydrocarbon feed reduces energy consumption of the fractional distillation column by about 5% or greater.

22. The method of claim 21, further comprising supplying the first ethylene fraction to one or more of a storage tank or an ethylene grid for supply to one or more ethylene consumers.

23. The method of claim 21, further comprising supplying the second ethylene fraction to a vinyl chloride monomer (VCM) process or similar process adapted to utilize ethylene in vapor phase and with an ethylene content of 99.90 wt. % or greater.

24. The method of claim 21, further comprising supplying the third ethylene fraction to a condenser; condensing at least a portion of the vent stream; and returning a recycled liquid stream containing ethylene to the fractional distillation column.
